(19) United States
(12) Patent Application Publication
(10) Pub. No.: US 2007/0191692 A1
(43) Pub. Date: Aug. 16, 2007

(54) Sleeping quality monitor system and method for monitoring a physiological signal

(76) Inventors: Yeh-Liang Hsu, Jhongli City (TW); Chang-Huei Wu, Jhongli City (TW); Chih-Ming Cheng, Jhongli City (TW); Hong-Xiang Ma, Johngli City (TW); Zhi-Wei Huan, Jhongli City (TW)

Correspondence Address: ROSENBERG, KLEIN & LEE, 3458 ELLICOTT CENTER DRIVE-SUITE 101, ELLICOTT CITY, MD, 21043, US

(21) Appl. No.: 11/582,422

(22) Filed: Oct. 18, 2006

(30) Foreign Application Priority Data
Feb. 14, 2006 (TW) 95104971

(51) Int. Cl. A61B 5/00; A61B 5/08; A61B 5/103

(52) U.S. Cl. 600/301; 128/903; 128/904; 600/300; 600/529; 600/549; 600/595; 128/920

(57) ABSTRACT

A sleeping quality monitor system and a method for monitoring a physiological signal monitors the sleeping quality of a person under test with long hours in daily life at home. The system comprises a distributed data server, at least one physiological signal sensor, and at least one client computer. The sensor is wired or wirelessly connected to the server. The server connects on Internet to the client computer. The sensor transmits the sensed physiological signals to the server to process, calculate, analyze, and store. By means of psychological signal data processing, the client computer further calculates and analyzes the psychological signal data stored in the server and allows an authorized reader to read the result. With the household sensor working with the server connecting to Internet, the system may be available in daily life at home to monitor the sleeping quality of the person under test with long hours.

### BACKGROUND OF THE INVENTION

1. Field of the Invention

This invention relates to a sleeping quality monitor system and a method for monitoring a physiological signal, and particularly to a sleeping quality monitor system applicable in daily life at home and suitable for monitoring the sleeping quality of a person under test with long hours and a method for monitoring a physiological signal thereof.

2. Description of Related Art

Generally, people spend one-third time in sleeping every day, but unhealthy sleep or insomnia is heavily harmful to them on physiology and psychology. Many people are troubled with sleep; even if they may easily fall asleep, the trouble of physiological reaction during sleep will also affect their physiological and psychological conditions and even threaten the lives, such as snoring. People who snore during sleep sometimes also stop breathing. Even if having enough sleeping time, yet due to awakening caused by many times of asphyxiation during sleep, people still feel insufficient sleep, which may seriously affect their brains and hearts on account of long-term anoxia.

A conventional sleeping quality monitor system uses a set of overnight polysomnography to monitor the times and types of breathe pause and shallow breath in a period of overnight sleep, the indices and times of anoxia, the variation of electrocardiogram, the airflow of mouth cavity and nasal cavity, the breathe running of chest and belly, oxygen content in blood, the times of snore as physiological conditions, and even extra instruments may be added to monitor the physiological items of the person under test based on his or her conditions. The system is sky-high precise and featured with wide monitor, so results given by the system are very accurate and close-knit, which is quite helpful to diagnosis on a disease. However, the system must be executed only in a medical institution or at a laboratory of an academic unit, so it is not suitable for long-term and universal monitor. Thus, only when being under the weather or infected with a disease, people in general leave for the medical institution for health check.

For this reason, in consideration of improving the defects described above, the inventor, having concentrated their studies and operating in coordination with academic theories, has finally provided this invention as a reasonable design and an effective improvement over the defects mentioned above.

### SUMMARY OF THE INVENTION

This invention is mainly to provide a sleeping quality monitor system and a method for monitoring a physiological signal, which may be used to monitor the sleeping quality of a person under test with long hours in daily life at home, detect the disturbance of sleeping of the person under test at an early date for speedy improvement, and thus prevent a disease caused by the disturbance of sleeping and the physiological abnormality during sleep.

In order to achieve the object, in this invention, a sleeping quality monitor system is provided, comprising a distributed data server, at least one physiological signal sensor, and at least one client computer. The physiological signal sensor is wired or wirelessly connected to the distributed data server and transmits monitored physiological signals to the distributed data server to process, calculate, analyze, and finally store. The client computer connects on Internet to the distributed data server, physiological signal data stored in the server may be read through the client computer on Internet, and then by means of physiological signal data processing, the physiological signal data is calculated and analyzed and then displayed on a PC screen. In this invention, a method for monitoring a physiological signal is provided.

In order to further know the features and technical means of this invention, refer to the detailed description according to this invention accompanied with drawings; however, the accompanied drawings are provided for reference and illustration only and are not limited to this invention.

### BRIEF DESCRIPTION OF THE DRAWINGS

FIG. 1 is a schematic view illustrating a system structure of an embodiment of this invention;

FIG. 2 is a flow chart of descriptive blocks of the system of the embodiment of this invention;

FIG. 3 is a schematic view illustrating the system structure of the other embodiment of this invention; and

FIG. 4 is a flow chart of descriptive blocks of the system of the other embodiment of this invention.

### DETAILED DESCRIPTION OF THE PREFERRED EMBODIMENTS

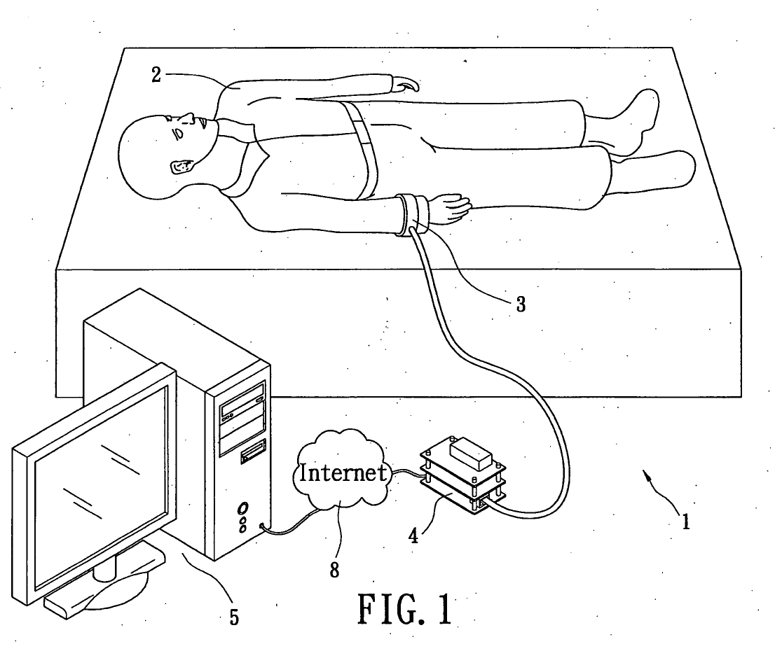

With reference to FIG. 1, a sleeping quality monitor system is provided in this invention to monitor the physiological signal of a person under test during sleep for determining his or her sleeping quality. The sleeping quality monitor system 1 comprises at least one physiological signal sensor 3, a distributed data server 4, and at least one client computer 5. The physiological signal sensor 3 may be wired through a transmission line or wirelessly connected through RS-232 as a wireless transmission module to the distributed data server 4. The client computer 5 is wired or wirelessly connected to Internet 8 and then connected to the distributed data server 4 on Internet. The physiological signal sensor 3 senses psychological signals, such as snore, breathe cycles per second, body movement or temperature and the like, and may be called a stertor sensor, a breathe cycle sensor, a body movement sensor, or a body temperature sensor, and they are household physiological signal sensor. On the other hand, the distributed data server 4 initially process, calculate, and analyze the psychological signals and then store them therein. And the data analyzed is also used to determine the physiological conditions of person under test. If finding abnormality, the distributed data server 4 will send an alarm message to a receiving end configured in advance. Further, the person under test may also send a call message actively, and when receiving the message, the server 4 will also send an alarm message to the receiving end. The relatives of the person under test, the person under test, or his or her doctor as an authorized agent may read the psychological signal data stored in the server 4 through the psychological signal data processed by the client computer 5, and after being further calculated and analyzed, the data of person under test may be read.

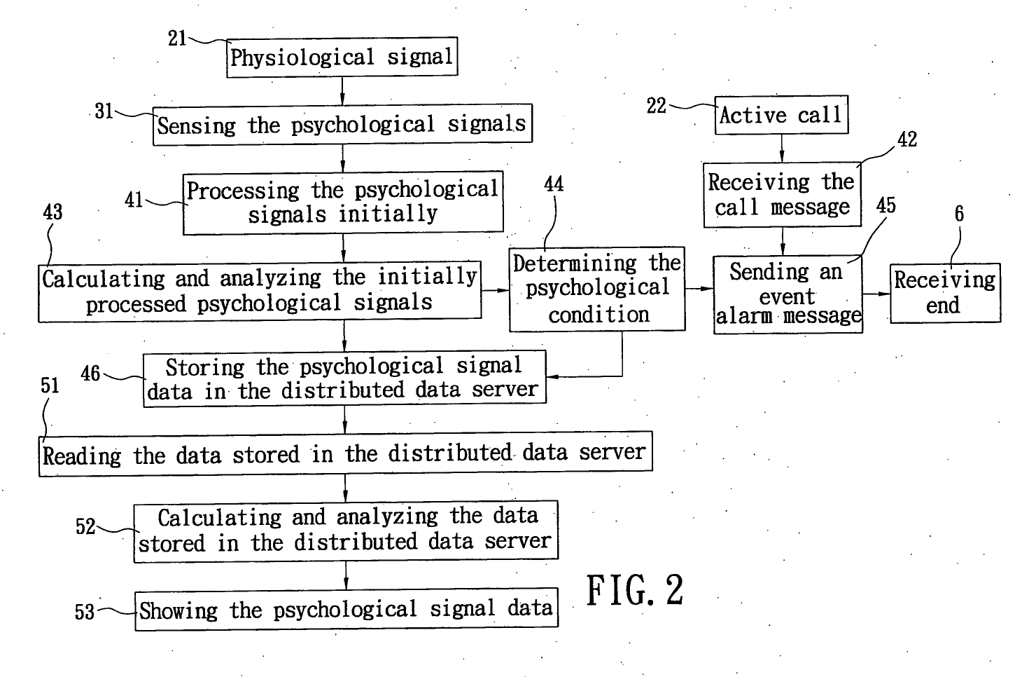

With reference to FIGS. 1 and 2, a method for monitoring the physiological signal is further provided in this invention to monitor the psychological signals 21 of the person under test 2 during sleep for determination of his or her sleeping quality. At first, the physiological signal sensor 3 senses the psychological signals 31. At the step, the physiological signal sensor 3 senses the psychological signals 21 of the person under test 2 and then transmits the psychological signals 21 to the distributed data server 4 to process psychological signals initially 41. Next, likewise in the distributed data server 4, the transmitted psychological signals 21 are calculated and analyzed 43. At the step, the distributed data server 4 in advance briefly calculates and analyzes the processed psychological signals 21. Next, the psychological signal data is stored in the distributed data server 46, and the data stored at this time is the original point data of the psychological signals and data having the psychological meaning. Then, the authorized reader reads the data stored in the distributed data server 51 by means of the psychological signal data processing in the client computer 5, and reads the psychological signal data stored in the distributed data server 4 on Internet 8. The means of the psychological signal data processing calculates and analyzes the data stored in the distributed data server 52 so that the reader may read what he or she want to in a chart. Finally, at the step of showing the psychological signal data 53, the chart of data is shown on a PC screen on which the authorized reader may read.

Further, after the step of calculating and analyzing the initially processed psychological signals 43, the distributed data server 4 stores the psychological signal data and meanwhile determines the result given from the pieces of data. At the step of determining the psychological condition 44, if it is found that the psychological signal of the person under test 2 is a condition in advance set up for an alarm, such as a higher temperature or psychological conditions of the person under test 2 that are same with long hours and regarded as an abnormal condition, such as no movement but breathe; in these two conditions, the distributed data server 4 sends an alarm message 45 to a receiving end 6 set up in advance. The alarm message may be sent to a mobile phone or a PDA working with a cell phone number or to an e-mail box. Besides, the person under test 2 may also send a call message in the manner of active call 22. From the distributed data server 4, the call message is received 42 and then sent to the receiving end 6.

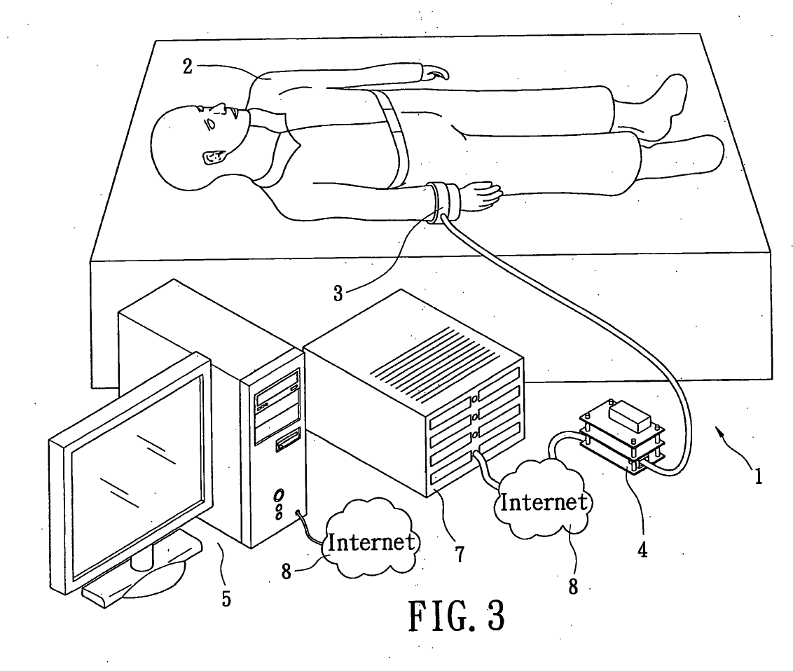

With reference to FIG. 3, in the embodiment, an application server 7 is mainly added to the sleeping quality monitor system 1. The application server 7 is wired or wirelessly connected to Internet 8 and then to the distributed data server 4. The application server 7 may read the psychological signal data stored in the distributed data server 4 by means of the psychological signal data processing and further calculate and analyze it and next stored it, and may manage the distributed data server 4 by means of system management and maintain and update the distributed data server 4. The client computer 5 is also connected on Internet to the application server 7. Browsing from the client computer 5 on Internet, the relatives of the person under test 2, the person under test 2, or his or her doctor as an authorized agent may read the psychological signal data stored in the application server.

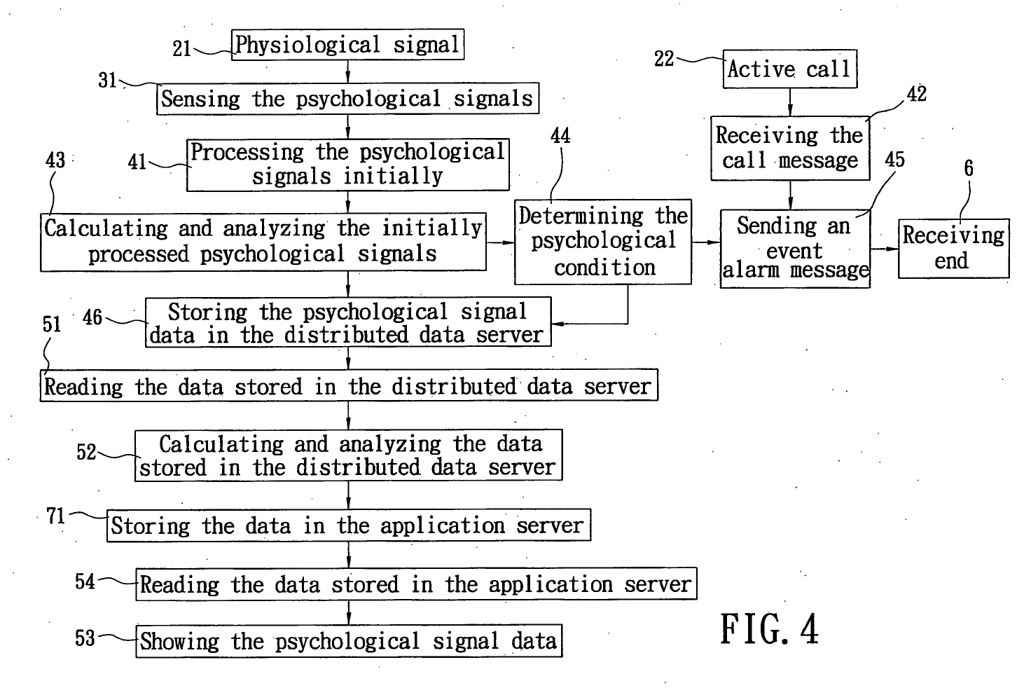

With reference to FIGS. 3 and 4, in the embodiment, a step 71 of storing the data in the application server 7 is added and a step 54 of reading the data stored in the application server from the client computer 5. Thus, by means of the psychological signal data processed by the application server 7, the data stored in the distributed data server 4 controls the application server 7 that reads the data 51 stored in the distributed data server 4, calculates and analyzes the data 52 stored in the distributed data server, and stores the data in the application server 71. At the three steps, the step 51 of reading data stored in the distributed data server is to read the psychological signal data stored in the distributed data server 4; also, for calculating and analyzing the data stored in the distributed data server 52 is to calculate and analyze the read psychological signal data stored in the distributed data server 4 for making various forms of charts. The chart is not made for the reader to directly read. The data is first stored in the application server 7 and the reader reads the data stored in the application server 54 via the client computer 5 on Internet by a mean of Internet navigating and watches the psychological signal data by a mean of showing the psychological signal data 53. The application server 7 is not confined in correspondence to a single distributed data server 4 and may meanwhile read the data stored in many distributed data servers 4 for achievement of management of a large number of data.

Further, in the two embodiments, by means of the psychological signal data processing and Internet navigating, real-time sensed data extracted by the psychological signal sensor 3 may be given so that the relatives or doctor of the person under test 2 may read real-time psychological variation and dispose of the abnormality.

In the embodiments, the physiological signal sensor 3 is used to sense psychological signals, such as snore, breathe cycles per second, body movement or temperature and the like. With the psychological signals, it is determined that the person under test 2 is awake or falls asleep or even deep asleep or shallow asleep at the present time, and thereby lay-up time, incubation period of sleep, sleep efficiency, times of awakening at the mid-night and the like as sleeping quality indices are given for evaluation of the sleeping quality of the person under test 2. With the household psychological signal sensor 3 working with the distributed data server 4 connecting to Internet 8, the sleeping quality may be monitored over a long period of time in daily life at home so that the disturbance of sleeping and the psychological abnormality during sleep may be found and improved early.

However, in the description mentioned above, only the preferred embodiments according to this invention are provided without limit to claims of this invention; all those skilled in the art without exception should include the equivalent changes and modifications as falling within the true scope and spirit of the present invention.

What is claimed is:

1. A sleeping quality monitor system used to monitor the psychological parameters of a person under test for determination of his or her sleeping quality, comprising:
a distributed data server;
at least one psychological signal sensor wired or wirelessly connected to the distributed data server; and
at least one client computer wired or wirelessly connected to the distributed data server.

2. The sleeping quality monitor system according to claim 1, wherein the distributed data server sends an alarm message to a receiving end.

3. The sleeping quality monitor system according to claim 2, wherein the receiving end is a mobile phone or a PDA working with a cell phone number, or an e-mail box.

4. The sleeping quality monitor system according to claim 1, wherein the psychological signal sensor is a stertor sensor, a breath cycle sensor, a body movement sensor, or a body temperature sensor.

5. The sleeping quality monitor system according to claim 1, wherein the distributed data server is connected to the psychological signal sensor via a transmission line.

6. The sleeping quality monitor system according to claim 1, wherein the physiological signal sensor is connected to the distributed data server via a RS-232 as a wireless transmission module for signal transmission.

7. The sleeping quality monitor system according to claim 1, wherein the means of psychological signal processing is provided in the client computer to calculate, analyze, and show the data stored in the distributed data server and extract real-time sensed data.

8. The sleeping quality monitor system according to claim 1, wherein the client computer connects to the distributed data server via Internet.

9. The sleeping quality monitor system according to claim 1, wherein an application server is included that is used to process and analyze data, show the data stored in the distributed data server, manage the distributed data server, and maintain and update system.

10. The sleeping quality monitor system according to claim 9, wherein the application server corresponds to a plurality of distributed data servers.

11. The sleeping quality monitor system according to claim 9, wherein the application server has means of processing psychological signal data and managing the system.

12. The sleeping quality monitor system according to claim 9, wherein the application server connects to the distributed data server via Internet.

13. The sleeping quality monitor system according to claim 9, wherein the application server connects to the client computer via Internet.

14. The sleeping quality monitor system according to claim 13, wherein the client computer has a mean of navigating Internet.

15. A method for monitoring a physiological signal that is executed in the distributed data server and the client computer to monitor the psychological parameters of the person under test for determination of his or her sleeping quality, comprising:
sensing a psychological signal of a person under test;
initially processing the sensed psychological signal of the person under test;
calculating and analyzing the initially processed psychological signal by the distributed data server;
determining a psychological condition, in which it is determined that a result given from whether the calculation of and analysis on the psychological signal is abnormal;
sending an event alarm message, in which the message is sent when the psychological condition is abnormal;
storing psychological signal data in the distributed data server, in which the data given from calculation and analysis and the result given from determination of the psychological condition are stored;
reading the data stored in the distributed data server, in which the psychological signal data stored in the distributed data server is read from the client computer;
calculating and analyzing the data stored in the distributed data server, in which the data stored in the distributed data server and read from the client computer is further calculated and analyzed; and
showing the psychological signal data, in which the resulted psychological signal data calculated and analyzed by the client computer is shown.

16. The method for monitoring a physiological signal according to claim 15, wherein the psychological signal sensed at the step of sensing the psychological signal is stertor, breathe cycles per second, and body movement or temperature.

17. The method for monitoring a physiological signal according to claim 15, wherein the abnormal condition of psychology determined is that set up in advance or staying in the same status for a long time.

18. The method for monitoring a physiological signal according to claim 15, wherein two steps of storing data in an application server and reading the data stored in an application server are included, wherein the step of storing the data in the application server is storing the result given from calculation of and analysis on the data stored in the distributed data server in the application server, the step of reading the data stored in the application server is reading the psychological signal data stored in the application server.
